(19) United States
(12) Patent Application Publication
(10) Pub. No.: US 2026/0272597 A1
(43) Pub. Date: Sep. 17, 2026

(54) SURGICAL INSTRUMENT COUNTING SYSTEM AND WORKING PLATFORM THEREOF

(71) Applicant: Wei-Chieh Ho, Taipei City (TW)

(72) Inventor: Wei-Chieh Ho, Taipei City (TW)

(21) Appl. No.: 19/569,210

(22) Filed: Mar. 17, 2026

Related U.S. Application Data
(60) Provisional application No. 63/773,152, filed on Mar. 17, 2025.

(51) Int. Cl. A61B 90/00; A61B 50/33; A61B 90/90

(52) U.S. Cl. CPC A61B 90/08; A61B 50/33; A61B 90/90; A61B 2090/0805

(57) ABSTRACT

A working platform for a surgical instrument counting system, comprising: a body portion; a first placement portion connected to the body portion; a second placement portion connected to the body portion; a connecting device for connecting to a plurality of first surgical instruments, each of the plurality of first surgical instruments having an annular portion; and a surgical instrument carrier loaded with a plurality of second surgical instruments, none of which has an annular portion; wherein the connecting device is removably connected to the first placement portion, and/or the surgical instrument carrier is removably connected to the second placement portion.

### TECHNICAL FIELD

The present disclosure relates to a surgical instrument counting system and a working platform thereof, and particularly to a surgical instrument counting system and a working platform thereof that can assist in the calculation of the number of surgical instruments.

### BACKGROUND

At present, the number of surgical instruments (such as scalpels or surgical scissors, but is not limited thereto) needs to be calculated and confirmed multiple times before and during the surgery. These calculation processes could not only exhaust the energy and efforts of the medical staff, but also considerably prolong the surgical time. Under these circumstances, there is a need for a surgical instrument counting system and a working platform thereof that can assist in the calculation of the number of surgical instruments.

### SUMMARY

To address the above-described problems, the idea of the present disclosure provides a surgical instrument counting system and a working platform thereof that can assist in the calculation of the number of surgical instruments and/or surgical supplies.

Based on the idea mentioned above, the present disclosure provides a working platform for a surgical instrument counting system, comprising: a body portion; a first placement portion connected to the body portion; a second placement portion connected to the body portion; a connecting device for connecting a plurality of first surgical instruments, each of the plurality of first surgical instruments having an annular portion; and a surgical instrument carrier loaded (or carried) with a plurality of second surgical instruments, none of the plurality of second surgical instruments having an annular portion; wherein the connecting device is removably connected to the first placement portion, and/or the surgical instrument carrier is removably connected to the second placement portion.

In a preferred embodiment of the present disclosure, the connecting device is a U-shaped rod.

In a preferred embodiment of the present disclosure, the first placement portion comprises a movable portion and a groove portion, the movable portion can move along the groove portion; the connecting device is removably connected to the movable portion of the first placement portion.

According to the purpose of the present disclosure, a surgical instrument counting system is further provided, comprising: a computing device that captures a first image of a plurality of first surgical instruments to generate a first image data, the plurality of the first surgical instruments each having an annular portion; wherein the computing device captures a second image of a plurality of second surgical instruments to generate a second image data, none of the plurality of second surgical instruments having an annular portion; wherein the computing device generates a first counting data based on the first image data, the first counting data indicating a first number of the plurality of first surgical instruments; wherein the computing device generates a second counting data based on the second image data, the second counting data indicating a second number of the plurality of second surgical instruments.

In a preferred embodiment of the present disclosure, the computing device comprises an image capturing portion; wherein the surgical instrument counting system further comprises a connecting device connected to the annular portion of each of the plurality of first surgical instruments; wherein, when the computing device captures the first image, the connecting device is disposed in front of the image capturing portion such that all of the plurality of first surgical instruments are positioned in front of the image capturing portion.

In a preferred embodiment of the present disclosure, the surgical instrument counting system further comprises: the working platform, wherein the working platform comprises a third placement portion connected to the body portion, and the computing device is connected to the third placement portion; wherein the computing device captures the first image of the plurality of first surgical instruments on the connecting device when the connecting device is removably connected to the first placement portion, thereby generating the first image data; wherein the computing device captures the second image of the plurality of second surgical instruments on the surgical instrument carrier when the surgical instrument carrier is removably connected to the second placement portion, thereby generating the second image data.

In a preferred embodiment of the present disclosure, the computing device comprises an image capturing portion; wherein the first placement portion comprises a first positioning mechanism, which positions the connecting device in front of the image capturing portion when the connecting device is connected to the first placement portion.

In a preferred embodiment of the present disclosure, the computing device comprises an image capturing portion; wherein the second placement portion comprises a second positioning mechanism, which positions the surgical instrument carrier in front of the image capturing portion when the surgical instrument carrier is connected to the second placement portion.

In a preferred embodiment of the present disclosure, the computing device comprises a first deep-learning module; wherein the computing device receives a first training data, and the computing device uses the first training data to train the first deep-learning module such that the first deep-learning module can generate the first counting data based on the first image data.

In a preferred embodiment of the present disclosure, the computing device comprises a second deep-learning module; wherein the computing device receives a second training data, and the computing device uses the second training data to train the second deep-learning module such that the second deep-learning module can generate the second counting data based on the second image data.

In a preferred embodiment of the present disclosure, the plurality of first surgical instruments comprises a third surgical instrument having a first label portion; wherein the first image data comprises a first label image data indicating the image of the first label portion; wherein the computing device generates a first identity data based on the first label image data, the first identity data is indicative of at least one of the type, material, usage number, and usage history information of the third surgical instrument.

In a preferred embodiment of the present disclosure, the plurality of second surgical instruments comprises a fourth surgical instrument having a second label portion; wherein the second image data includes a second label image data, which is indicative of the image of the second label portion; wherein the computing device generates a second identity data based on the second label image data, wherein the second identity data is indicative of at least one of the type, material, usage number, and usage history information of the fourth surgical instrument.

In a preferred embodiment of the present disclosure, the computing device comprises a display portion; wherein, when the computing device generates the first counting data based on the first image data, the display portion displays the first image data and/or the first counting data; wherein, when the computing device generates the second counting data based on the second image data, the display portion displays the second image data and/or the second counting data.

The various aspects of the present disclosure stated above, together with other aspects of the present disclosure, will be clarified further hereinafter based on the following detailed descriptions of the non-restrictive embodiments and their accompanying figures.

### BRIEF DESCRIPTION OF THE DRAWINGS

FIG. 1 is a schematic diagram showing an embodiment of a working platform for a surgical instrument counting system.

FIG. 2 is a schematic diagram showing an embodiment of the working platform for a surgical instrument counting system, after a connecting device and a surgical instrument carrier are removed.

FIG. 3 is a schematic diagram showing an embodiment of a computing device.

FIG. 4 is a schematic diagram showing an embodiment of a computing device

FIG. 5 is a partially magnified schematic diagram of the first placement portion located in area B of FIG. 2.

FIG. 6 is a system architecture diagram of an embodiment of a computing device.

FIG. 7 is a schematic diagram of a connecting device connected to a plurality of first surgical instruments.

FIG. 8 is a schematic diagram of a surgical instrument carrier loaded with a plurality of second surgical instruments.

FIG. 9 is a schematic diagram of a connecting device connected to a first placement portion.

FIG. 10 is a schematic diagram of a surgical instrument carrier connected to a second placement portion.

FIG. 11 is a schematic diagram showing an embodiment of a first image data and a first counting data.

FIG. 12 is a schematic diagram showing an embodiment of a second image data and a second counting data.

### DETAILED DESCRIPTION

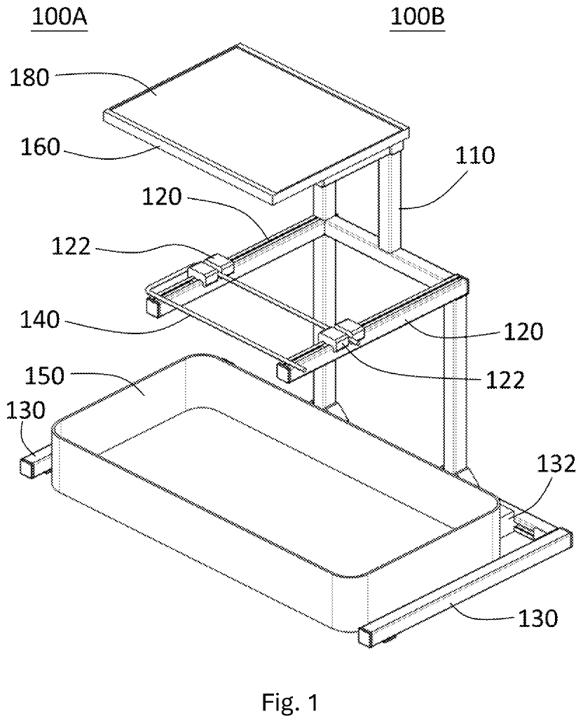

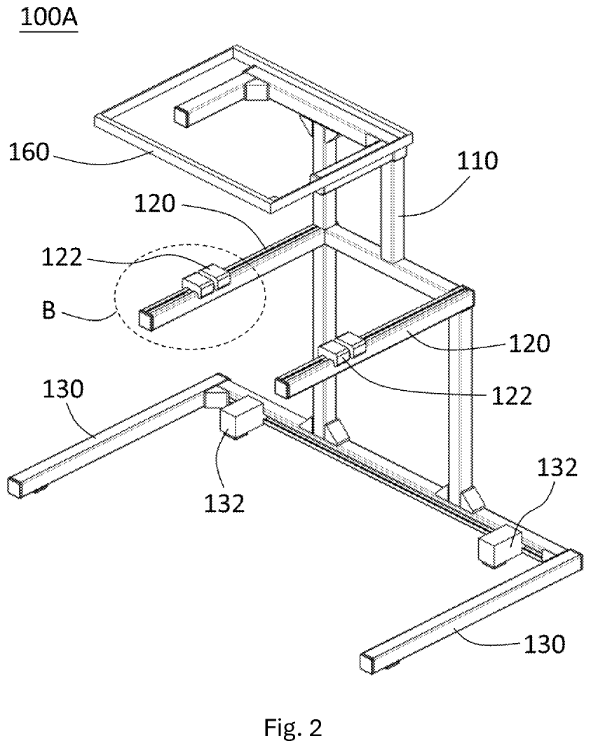

Please refer to FIGS. 1 and 2 at the same time, wherein FIG. 1 shows a schematic diagram of an embodiment of a working platform for a surgical instrument counting system according to the present disclosure; FIG. 2 shows a schematic diagram of a working platform for a surgical instrument counting system according to the present disclosure after removing a connecting device and a surgical instrument carrier. As shown in FIGS. 1 and 2, the working platform 100A for the surgical instrument counting system comprises a body portion 110, a first placement portion 120, a second placement portion 130, a connecting device 140, and a surgical instrument carrier 150. Both the first placement portion 120 and the second placement portion 130 are connected to the body portion 110. The connecting device 140 can be removably connected to the first placement portion when necessary (for example, the connecting device 140 is removably placed or clamped onto the first placement portion, but is not limited thereto), and the connecting device 140 can be used to connect to a plurality of first surgical instruments. The surgical instrument carrier 150 can be removably connected to the second placement portion 130 when necessary (for example, the surgical instrument carrier 150 can be removably abutted, clamped, or placed at the second placement portion, but is not limited thereto), and the surgical instrument carrier 150 can be used to load a plurality of second surgical instruments.

Preferably, each of the plurality of first surgical instruments connected to the connecting device 140 has an annular portion (which may be referred to as an annular grip portion). Preferably, the medical staff may put their fingers through the annular portion, thus facilitating the operation of the first surgical instrument), while none of the plurality of second surgical instruments loaded on the surgical instrument carrier 150 has an annular portion. Preferably, when the connecting device 140 is connected to the plurality of first surgical instruments, the connecting device is connected to each of the annular portions of the plurality of first surgical instruments. Preferably, when the connecting device 140 is connected to a plurality of first surgical instruments, the connecting device 140 passes through each of the annular portions of the plurality of first surgical instruments, thereby aligning or securing the plurality of first surgical instruments (for example, arranging the plurality of annular surgical instruments in an array). The connecting device 140 may be, for example, a U-shaped rod, but is not limited thereto. It should be understood that the connecting device 140 may also be a rod in other shapes as needed (for example, a C-shaped rod or an elongated rod, but is not limited thereto), or the connecting device 140 may also be other types of connecting devices as needed. Preferably, the surgical instrument carrier may be, for example, a surgical instrument basket or a surgical instrument tray, but is not limited thereto.

In the embodiment shown in FIG. 1, the working platform 100A may further comprise a third placement section 160, which is connected to the body portion 110. The third placement section 160 can be used to connect to a computing device 180 (for example, the third placement section 160 can be used to accommodate the computing device 180, but is not limited thereto). In an embodiment, the surgical instrument counting system 100B of the present disclosure may comprise a working platform 100A and a computing device 180. The computing device 180 may be, for example, a computer, a mobile device, a mobile phone, or a tablet computer, but is not limited thereto. The computing device 180 may also be other devices comprising an image capturing function and/or a data processing function as needed. Preferably, the computing device 180 may comprise one or more processors, and implement the functions of the computing device 180 in a manner that hardware and software collaborate with each other. Preferably, the computing device 180 implements the functions of each module (such as the first deep-learning module and the second deep-learning module described later) in a manner that hardware and software collaborate with each other. Preferably, the computing device 180 implements the functions of the image capturing portion and/or the display portion in a manner that hardware and software collaborate with each other.

In an embodiment, when the connecting device 140 is removably connected to the first placement portion 120, the computing device 180 can capture the image of a plurality of first surgical instruments on the connecting device 140 (herein referred to as the first image), thereby generating a first image data. The computing device 180 can then generate a first counting data based on the first image data, wherein the first counting data is indicative of the number of the first surgical instrument (herein referred to as a first number). Furthermore, when the surgical instrument carrier 150 is removably connected to the second placement portion 130, the computing device 180 can capture the image of the plurality of second surgical instruments on the surgical instrument carrier 150 (herein referred to as a second image), thereby generating a second image data. The computing device 180 can then generate the second counting data based on the second image data, wherein the second counting data is indicative of a second number of the second surgical instruments (referred to as a second number). This allows the computing device 180 to quickly calculate a number of the plurality of first surgical instruments and a plurality of the second surgical instruments, without the need to count the number of the first surgical instrument and the number of the second surgical instrument manually. Furthermore, using the computing device 180 to calculate the number of the surgical instruments can improve counting accuracy and avoid miscalculations caused by manual calculation. Overall, the surgical instrument counting system and its working platform thereof will significantly reduce the time required to count surgical instruments and improve accuracy of calculations.

Preferably, the computing device 180 can capture the first image and the second image separately. For example, the user can first connect the plurality of first surgical instruments to the connecting device 140, then place the connecting device 140 on the first placement portion 120, and then allow the computing device 180 to capture the first image of the plurality of first surgical instruments on the connecting device 140. Afterwards, the user can load or place the plurality of second surgical instruments onto the surgical instrument carrier 150, and place the surgical instrument carrier 150 on the second placement portion. Next, the user may allow the computing device 180 to capture the second image of the plurality of second surgical instruments on the surgical instrument carrier 150. Certainly, the user may capture the second image first before capturing the first image as needed.

Preferably, when capturing the first image of the plurality of first surgical instruments, the surgical instrument carrier 150 may not be placed on the second placement portion 130. This avoids the first image including the image of the second surgical instruments, which significantly increases the difficulty for the computing device 180 to determine (or count) the number of the first surgical instruments. Likewise, when capturing the second image of the plurality of second surgical instruments, the connecting device 140 may not be placed on the first placement portion 120. This avoids the second image including the image of the first surgical instruments, which significantly increases the difficulty for the computing device 180 to determine (or count) the number of the second surgical instruments. It should be understood that, by placing the first surgical instruments and the second surgical instruments separately, and by capturing the first image of the first surgical instruments and the second image of the second surgical instruments separately, the complexity of the images can be significantly reduced, and the accuracy of the computing device in determining (or counting) the number of surgical instruments (including the first surgical instruments and the second surgical instruments) can be significantly improved. However, it should be understood that the user may choose not to separately place the first surgical instruments and the second surgical instruments as needed, and choose to capture the first image and the second image simultaneously in order to save time.

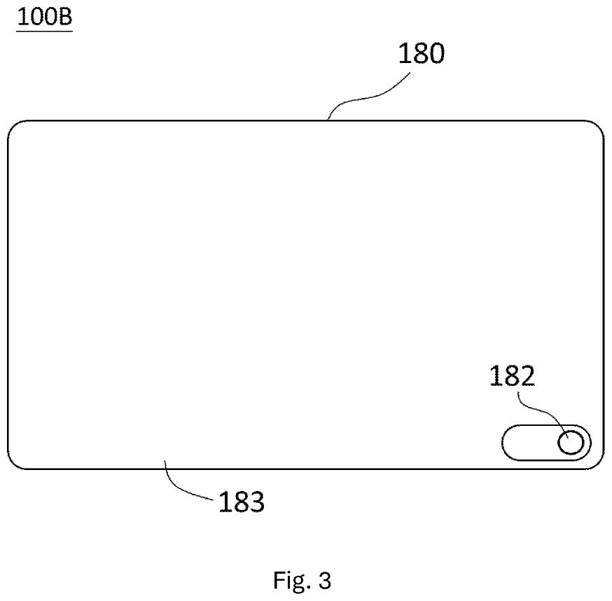

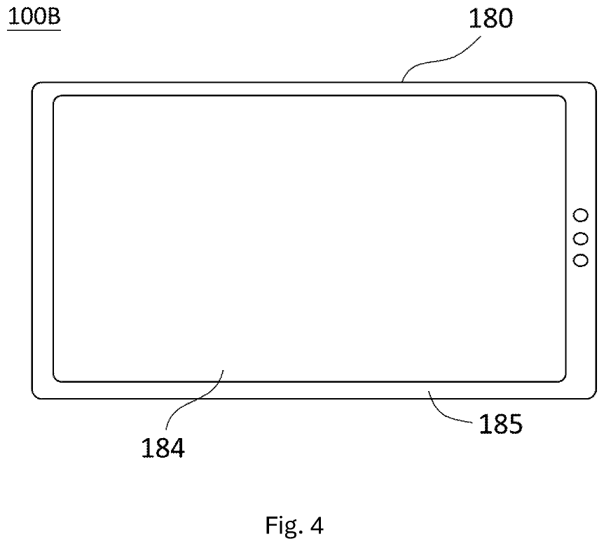

Please refer to the embodiments shown in FIGS. 3 and 4, wherein each is a schematic diagram showing an embodiment of a computing device. In the embodiments shown in FIGS. 3 and 4, the computing device 180 may comprise an image capturing portion 182 and a display portion 184. The image capturing portion 182 can capture images (preferably, the computing device 180 uses the image capturing portion 182 to capture the first image data and/or the second image data), and the display portion 184 can display the images. Preferably, the computing device 180 may have a first surface 183 and a second surface 185, and the first surface 183 and the second surface 185 are opposite to each other. The image capturing portion 182 is disposed on the first surface 183, while the display portion 184 is disposed on the second surface 185 that is opposite to the first surface 183. Preferably, when the third placement portion 160 is connected to the computing device 180, the image capturing portion 182 of the computing device 180 faces toward the first placement portion 120 and/or the second placement portion 130. Preferably, when the third placement portion 160 is connected to the computing device 180, the image capturing portion 182 of the computing device 180 faces toward the connecting device 140 on the first placement portion 120, or faces toward the surgical instrument carrier 150 on the second placement portion 130.

Preferably, when the computing device 180 generates the first counting data based on the first image data, the display portion 184 can display the first image data and/or the first counting data. Thus, the user can determine whether the computing device 180 has accurately captured the image of the plurality of first surgical instruments based on the first image data displayed on the display portion 184. The user can also determine the number of the plurality of first surgical instruments based on the first counting data displayed on the display portion 184. Furthermore, when the computing device 180 generates the second counting data based on the second image data, the display portion 184 can display the second image data and/or the second counting data. Thus, the user can determine whether the computing device 180 has accurately captured the image of the plurality of second surgical instruments based on the second image data displayed on the display portion 184. The user can also determine the number of the plurality of second surgical instruments based on the second counting data displayed on the display portion 184.

In an embodiment, the surgical instrument counting system 100B of the present disclosure may comprise a computing device 180. In this case, the surgical instrument counting system 100B may either without the aforementioned working platform 100A, or without the body portion, the first placement portion, and the second placement portion of the working platform 100A. The computing device 180 may capture the first image of the plurality of first surgical instruments to generate the first image data, each of the plurality of first surgical instruments having an annular portion. The computing device 180 may also capture the second image of the plurality of the second surgical instruments to generate the second image data, none of the plurality of second surgical instruments having an annular portion. The computing device 180 may generate the first counting data based on the first image data, the first counting data indicating the first number of the plurality of first surgical instruments. The computing device 180 may also generate the second counting data based on the second image data, the second counting data indicating the second number of the plurality of the second surgical instruments. Preferably, the computing device 180 captures the first image and the second image separately. It should be understood that, by capturing the first image of the first surgical instruments and the second image of the second surgical instruments separately, the complexity of the images can be significantly reduced, and the accuracy of the computing device in determining the number of surgical instruments (including the first surgical instruments and the second surgical instruments) can be significantly improved. However, it should be understood that the user may choose not to separately place the first surgical instruments and the second surgical instruments as needed, and choose to capture the first image and the second image simultaneously in order to save time.

Preferably, the surgical instrument counting system 100B may further comprise a connecting device (such as the aforementioned connecting device 140), which can connect to the annular portion of each of the plurality of first surgical instruments. When the computing device 180 captures the first image of the plurality of first surgical instruments, the connecting device is positioned in front of the image capturing portion 182 of the computing device 180 (the connecting device does not need to connect to the first placement portion of the working platform at the same time) such that the plurality of first surgical instruments are all positioned in front of the image capturing portion 182. In an embodiment, the surgical instrument counting system 100B may further comprise a surgical instrument carrier (such as the aforementioned surgical instrument carrier 150), which can load a plurality of second surgical instruments. When the computing device 180 captures the second images of the plurality of second surgical instruments, the surgical instrument carrier is positioned in front of the image capturing portion 182 of the computing device 180 (the surgical instrument carrier does not need to connect to the second placement portion of the working platform at the same time) such that the plurality of second surgical instruments are all positioned in front of the image capturing portion 182.

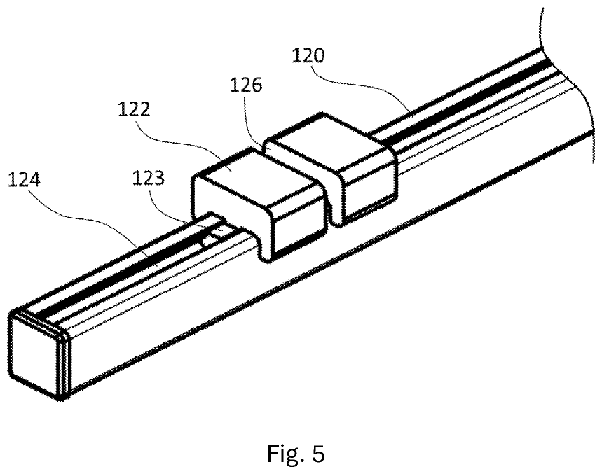

Next, please refer to FIGS. 1-5, wherein FIG. 5 is a partially magnified schematic diagram of the first placement portion 120 located in area B of FIG. 2. In the embodiments shown in FIGS. 1-5, the first placement portion 120 may comprise a movable portion 122 and a groove portion 124. The movable portion 122 may move along the groove portion 124, and the connecting device 140 is removably connected to the movable portion 122 of the first placement portion 120. Thus, when the connecting device 140 is connected to the movable portion 122 of the first placement portion 120, the connecting device 140 can move relative to the groove portion 124 along with the movable portion 120, thereby allowing the user to adjust the relative position between the connecting device 140 and the body portion 110, and/or adjust the relative position between the connecting device 140 and the computing device 180 connected to the third placement portion 160. In an embodiment, the movable portion 122 may have a guiding portion 123 disposed in the groove portion 124 to guide the movable portion 122 to move along the groove portion 124. Preferably, the guide portion 123 is snapped in the groove portion 124 to prevent the movable portion 122 completely disengaging from the groove portion 124.

Preferably, the first placement portion 120 may have a first positioning mechanism 126. When the connecting device 140 is connected to the first placement portion 120, the first positioning mechanism 126 positions the connecting device 140 in front of the image capturing portion 182 (for example, the first positioning mechanism 126 can secure the connecting device 140 in front of the image capturing portion 182). Thus, when the connecting device 140 is placed at the first placement portion 120, the computing device 180 or its image capturing portion 182 can directly capture the first image of the plurality of first surgical instruments. Preferably, the first positioning mechanism 126 is disposed on the movable portion 122. Preferably, the first positioning mechanism 126 is disposed at the positioning groove portion on the movable portion 122, and the connecting device 140 can be removably connected to (for example, snapped into) the positioning groove portion.

Preferably, the second placement portion may have a second positioning mechanism 132, when the surgical instrument carrier 150 is connected to the second placement portion 130, the second positioning mechanism 132 allows the surgical instrument carrier 150 to be positioned in front of the image capturing portion 182 (for example, the second positioning mechanism 132 can abut against the surgical instrument carrier 150 to position the surgical instrument carrier 150 in front of the image capturing portion 182). Thus, when the surgical instrument carrier 150 is disposed at the second placement portion 130, the computing device 180 or its image capturing portion 182 can directly capture the second image of the plurality of surgical instruments that have no annular portions.

Preferably, the third placement portion 160 may be disposed above the first placement portion 120 and the second placement portion 130. Thus, when the computing device 180 is connected to the third placement portion 160, the computing device 180 or its image capturing portion 182 can face toward the first placement portion 120 and/or the second placement portion 130. Preferably, the third placement portion 160 is configured such that, when the computing device 180 is connected to the third placement portion 160, and the connecting device 140 is connected to the first placement portion 120, the image capturing portion 182 of the computing device 180 faces toward the connecting device 140 on the first placement portion 120. Furthermore, the third placement section 160 is configured such that, when the computing device 180 is connected to the third placement section 160, and the surgical instrument carrier 150 is connected to the second placement section 130, the image capturing section 182 of the computing device 180 faces toward the surgical instrument carrier 150 on the second placement section 130.

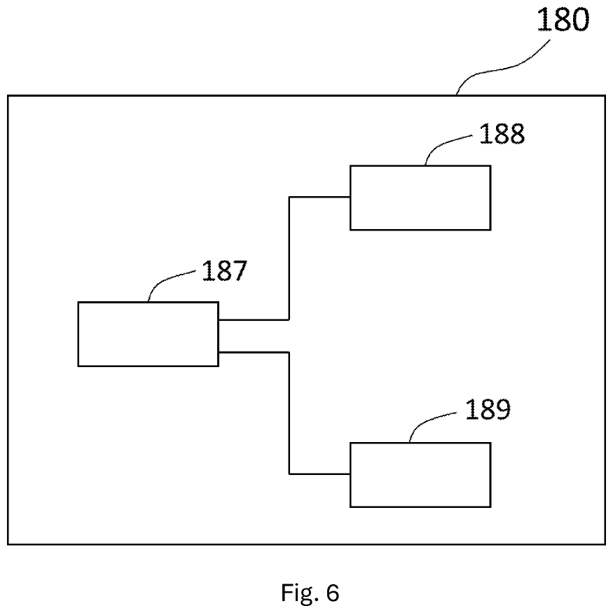

Please refer to FIG. 6, which shows a system architecture diagram of an embodiment of a computing device. As shown in FIG. 6, the computing device 180 may comprise a database 187, a first deep-learning module 188, and a second deep-learning module 189 (as shown in FIG. 6). The first deep-learning module 188 and the second deep-learning module 189 have access to the database 187 (preferably, the image capturing portion 182 is communicatively connected to the database 187). The computing device 180 can store the types of data (for example, the first image data, the second image data, the first counting data, the second counting data, the various training data, and so on, but not limited thereto) into the database 187. The computing device 180 can receive a first training data, and it can use the first training data to train the first deep-learning module 188 such that the first deep-learning module 188 can generate a first counting data based on the first image data. Furthermore, the computing device 180 can receive a second training data, and it can use this second training data to train a second deep-learning module 189 such that the second deep-learning module 189 can generate the second counting data based on the second image data. Preferably, the computing device 180 uses the first deep-learning module 188 to generate the first counting data based on the first image data. Furthermore, the computing device 180 uses the second deep-learning module 189 to generate the second counting data based on the second image data. Preferably, the first deep-learning module 188 and the second deep-learning module 189 are different modules. However, the user may also configure the first deep-learning module and the second deep-learning module to be the same module as needed.

Preferably, the first training data may include a first training image data and a number data, wherein the first training image data includes images of a plurality of fifth surgical instruments, each of which has an annular portion (i.e., all of the plurality of fifth surgical instruments are surgical instruments with an annular portion, and the annular portion of the fifth surgical instruments may be referred to as a second annular portion). The number data is indicative of the number of the plurality of fifth surgical instruments. Preferably, the computing device may use multiple sets of different first training data to train the first deep-learning module 188, thereby allowing the first deep-learning module 188 to generate the first counting data based on the first image data.

Preferably, the second training data may include a second training image data and a second number data, wherein the second training image data includes images of a plurality of sixth surgical instruments, none of which has an annular portion (i.e., all of the plurality of sixth surgical instruments are surgical instruments with an annular portion) . The second number data is indicative of the number of the plurality of sixth surgical instruments. Preferably, the computing device 180 can use multiple sets of different second training data to train the second deep-learning module 189, thereby allowing the second deep-learning module 189 to generate the second counting data based on the second image data.

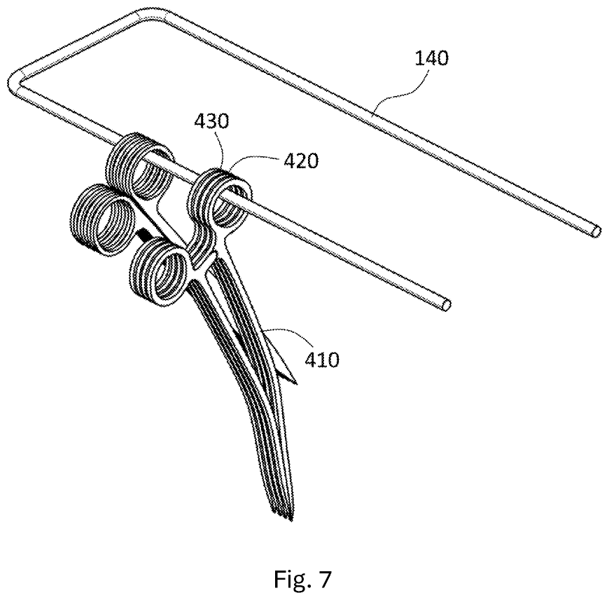

Please refer to FIG. 7, which is a schematic diagram of a connecting device connected to a plurality of first surgical instruments. As shown in the embodiment of FIG. 7, the connecting device 140 can connect to a plurality of first surgical instruments (for example, the first surgical instruments 410-430 in the figures), wherein all of the first surgical instruments 410-430 are surgical instruments with an annular portion (which may be referred to as a first annular portion), and therefore the first surgical instruments may be referred to as surgical instruments with an annular portion. The first surgical instruments may be, for example, surgical scissors with an annular portion, or surgical forceps with an annular portion, but is not limited thereto. The annular portion of the surgical scissors/surgical forceps may be disposed at an end of the gripping portion of the surgical scissors/surgical forceps. Thus, the medical staff can put their fingers through the annular portion to operate the surgical scissors/forceps with ease.

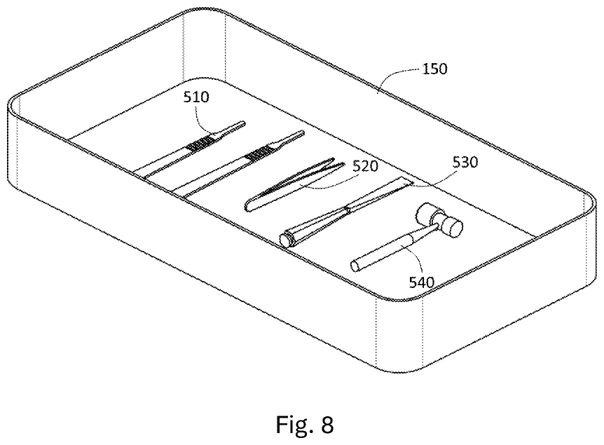

Please refer to FIG. 8, which is a schematic diagram of a surgical instrument carrier loaded with a plurality of second surgical instruments. As shown in the embodiment of FIG. 8, the surgical instrument carrier 150 can be loaded with a plurality of second surgical instruments (for example, the second surgical instruments 510-540 in the figure), wherein all of the second surgical instruments 510-540 are surgical instruments without an annular portion; therefore, the second surgical instruments can be referred to as surgical instruments without an annular portion. The second surgical instruments may be, for example, scalpels and surgical clips without an annular portion, but is not limited thereto.

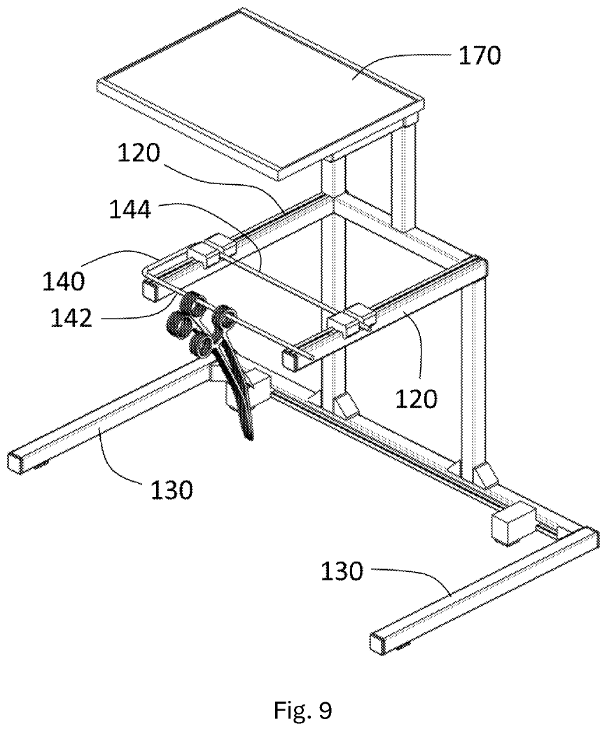

Please refer to FIG. 9, which is a schematic diagram of a connecting device connected to a first placement portion. As shown in the embodiment of FIG. 9, the plurality of first surgical instruments with an annular portion are connected to a connecting device 140, and the connecting device 140 is connected to the first placement portion 120, while the surgical instrument carrier 150 is not connected to the second placement portion 130. Preferably, under these circumstances, the computing device 180 can capture the first image of the plurality of first surgical instruments to generate the first image data. It should be understood that, FIG. 9 merely serves as an example here; the plurality of first surgical instruments are not limited to be connected to a first side 142 (or referred as first side rod 142) of the connecting device 140, but can be connected to a second side 144 (or referred as second side rod 144) of the connecting device 140 instead if needed. Preferably, the plurality of first surgical instruments should be connected to the same side of the connecting device 140 (for example, the first side 142 or the second side 144). However, the user may, depending on the needs (for example, when the number of first surgical instruments is high and it is impossible to connect all of the first surgical instruments to the same side of the connecting device 140), dispose the plurality of first surgical instruments on both of the first side 142 and the second side 144.

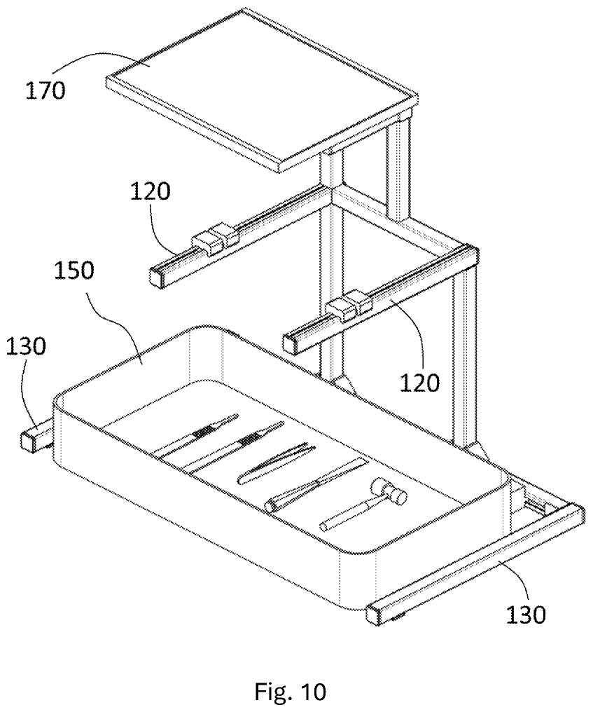

Please refer to FIG. 10, which is a schematic diagram of a surgical instrument carrier connected to a second placement portion. As shown in the embodiment of FIG. 10, a plurality of second surgical instruments without an annular portion are placed in the surgical instrument carrier 150, which is connected to a second placement portion 130, and the connecting device 140 is not connected to a first placement portion 120. Preferably, under these circumstances, the computing device 180 can capture the second image of the plurality of second surgical instruments to generate the second image data.

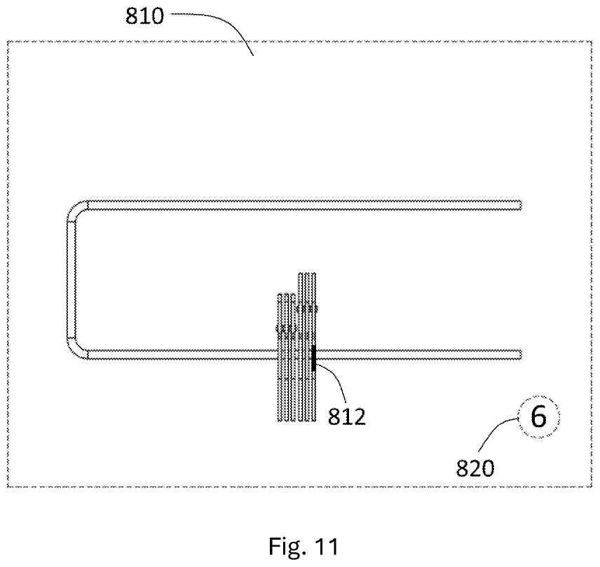

Please refer to FIG. 11, which is a schematic diagram of an embodiment of a first image data and a first counting data. As shown in the embodiment of FIG. 11, the plurality of first surgical instruments may include a third surgical instrument having a first label portion. The first image data 810 generated by the computing device 180 includes a first label image data 812, and the first label image data 812 is indicative of the image of a first label portion of the third surgical instrument. The computing device 180 can generate a first identity data based on the first label image data 812, wherein the first identity data is indicative of at least one of the type, material, usage number, and usage history information (preferably, the usage history information is indicative of which surgeries that the third surgical instrument have been used in) of the third surgical instrument. The first label portion may, for example, be a notch or label (for example, a one-dimensional barcode, a two-dimensional barcode, RFID, and so on, but is not limited thereto) provided on the third surgical instrument.

Preferably, when the third surgical instrument is placed on the connecting device 140, the first label portion faces toward the computing device 180 and/or the image capturing portion 182 of the computing device 180 (for example, the computing device 180 is disposed above the first placement portion 120 and/or the connecting device 140, and the first label portion faces toward the computing device 180 located above it) such that the computing device 180 can capture the image of the first label portion. Preferably, when the third surgical instrument are placed on the connecting device 140, the connecting device 140 allows the first label portion to face toward the computing device 180 and/or the image capturing portion 182 of the computing device 180 such that the computing device 180 can capture the image of the first label portion. For example, the first label portion may be disposed at the rear end of the annular portion of the third surgical instrument. When the connecting device 140 passes through the annular portion of the third surgical instrument, the first label portion disposed at the rear end of the annular portion will turn upward and face toward the computing device 180 and/or the image capturing portion 182 of the computing device 180 because of the heavier front end of the third surgical instrument. Preferably, when the computing device 180 displays the first image data 810 through the display portion 184, the first counting data 820 can also be displayed on the screen. Thus, the user can determine (confirm) the first image data 810 and the first counting data 820 through the display portion 184.

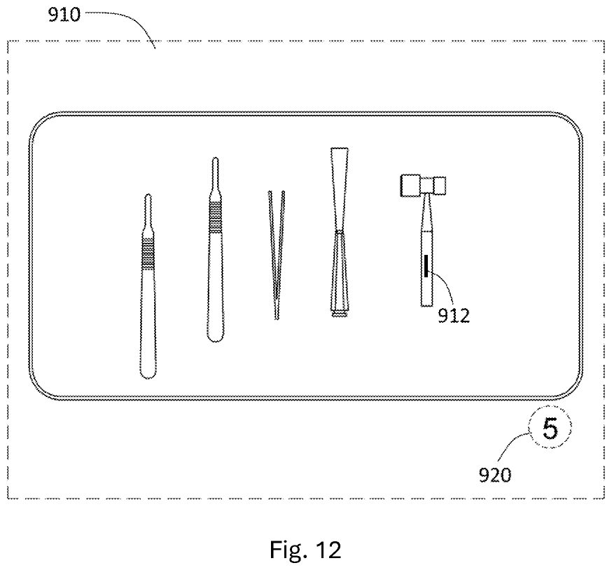

Please refer to FIG. 12, which is a schematic diagram of an embodiment of a second image data and a second counting data. As shown in the embodiment of FIG. 12, a plurality of second surgical instruments may comprise a fourth surgical instrument having a second label portion. The second image data 910 generated by the computing device 180 includes a second label image data 912, which is indicative of the image of the second label portion of the fourth surgical instrument. The computing device 180 may generate a second identity data based on the second label image data 912, wherein the second identity data is indicative of at least one of the type, material, usage number, and usage history information (preferably, the usage history information is indicative of which surgeries that the fourth surgical instrument have been used in) of the fourth surgical instrument. The second label portion may, for example, be a notch or label (for example, a one-dimensional barcode, a two-dimensional barcode, RFID, and so on, but is not limited thereto) provided on the fourth surgical instrument.

Preferably, when the fourth surgical instrument is placed on the surgical instrument carrier 150, the second label portion faces toward the computing device 180 and/or the image capturing portion 182 of the computing device 180 (for example, the computing device 180 is positioned above the second placement portion 130 and/or the surgical instrument carrier 150, and the second label portion faces upward) such that the computing device can capture the image of the second label portion. Preferably, when the computing device 180 displays the second image data 910 through the display portion 184, the second counting data 920 can also be displayed on the screen. Thus, the user can determine the second image data 910 and the second counting data 920 through the display portion 184.

The surgical instrument counting system and working platform thereof in the present disclosure is described above and depicted in the drawings. However, it should be noted that those various embodiments above can be implemented alone or in combination of one or more embodiments. It should be understood that the embodiments of the present disclosure are merely exemplary in nature. Various changes may be made to the embodiments of the present disclosure without departing from the spirit and scope of the claims of the present disclosure, and such changes shall be covered by the scope of the claims of the present disclosure. Therefore, the embodiments of the present disclosure are not intended to restrict the claimed invention, and the actual scope and spirit of the present disclosure is disclosed in the appended claims.

What is claimed is:

1. A working platform for a surgical instrument counting system, comprising:
a body portion;
a first placement portion connected to the body portion;
a second placement portion connected to the body portion;
a connecting device for connecting a plurality of first surgical instruments, each of the plurality of first surgical instruments having an annular portion; and
a surgical instrument carrier loaded with a plurality of second surgical instruments, none of the plurality of second surgical instruments having an annular portion;
wherein the connecting device is removably connected to the first placement portion, and/or the surgical instrument carrier is removably connected to the second placement portion.

2. The working platform of claim 1, wherein the connecting device is a U-shaped rod.

3. The working platform of claim 1, wherein the first placement portion comprises a movable portion and a groove portion, the movable portion can move along the groove portion;
wherein the connecting device is removably connected to the movable portion of the first placement portion.

4. A surgical instrument counting system, comprising:
a computing device that captures a first image of a plurality of first surgical instruments to generate a first image data, the plurality of the first surgical instruments each having an annular portion;
wherein the computing device captures a second image of a plurality of second surgical instruments to generate a second image data, none of the plurality of second surgical instruments having an annular portion;
wherein the computing device generates a first counting data based on the first image data, the first counting data indicating a first number of the plurality of first surgical instruments;
wherein the computing device generates a second counting data based on the second image data, the second counting data indicating a second number of the plurality of second surgical instruments.

5. The surgical instrument counting system of claim 4, wherein the computing device comprises an image capturing portion;
wherein the surgical instrument counting system further comprises a connecting device connected to the annular portion of each of the plurality of first surgical instruments;
wherein, when the computing device captures the first image, the connecting device is disposed in front of the image capturing portion such that all of the plurality of first surgical instruments are positioned in front of the image capturing portion.

6. The surgical instrument counting system of claim 4, further comprising: any working platform of claims 1-3, wherein the working platform comprises a third placement portion connected to the body portion, and the computing device is connected to the third placement portion;
wherein the computing device captures the first image of the plurality of first surgical instruments on the connecting device when the connecting device is removably connected to the first placement portion, thereby generating the first image data;
wherein the computing device captures the second image of the plurality of second surgical instruments on the surgical instrument carrier when the surgical instrument carrier is removably connected to the second placement portion, thereby generating the second image data.

7. The surgical instrument counting system of claim 6, wherein the computing device comprises an image capturing portion;
wherein the first placement portion comprises a first positioning mechanism, which positions the connecting device in front of the image capturing portion when the connecting device is connected to the first placement portion.

8. The surgical instrument counting system of claim 6, wherein the computing device comprises an image capturing portion;
wherein the second placement portion comprises a second positioning mechanism, which positions the surgical instrument carrier in front of the image capturing portion when the surgical instrument carrier is connected to the second placement portion.

9. The surgical instrument counting system of claim 4, wherein the computing device comprises a first deep-learning module;
wherein the computing device receives a first training data, and the computing device uses the first training data to train the first deep-learning module such that the first deep-learning module can generate the first counting data based on the first image data.

10. The surgical instrument counting system of claim 4, wherein the computing device comprises a second deep-learning module;
wherein the computing device receives a second training data, and the computing device uses the second training data to train the second deep-learning module such that the second deep-learning module can generate the second counting data based on the second image data.

11. The surgical instrument counting system of claim 4, wherein the plurality of first surgical instruments comprises a third surgical instrument having a first label portion;
wherein the first image data comprises a first label image data indicating the image of the first label portion;
wherein the computing device generates a first identity data based on the first label image data, and the first identity data is indicative of at least one of the type, material, usage number, and usage history information of the third surgical instrument.

12. The surgical instrument counting system of claim 4, wherein the plurality of second surgical instruments comprises a fourth surgical instrument having a second label portion;
wherein the second image data includes a second label image data, which is indicative of the image of the second label portion;
wherein the computing device generates a second identity data based on the second label image data, wherein the second identity data is indicative of at least one of the type, material, usage number, and usage history information of the fourth surgical instrument.

13. The surgical instrument counting system of claim 4, wherein the computing device comprises a display portion;
wherein, when the computing device generates the first counting data based on the first image data, the display portion displays the first image data and/or the first counting data;
wherein, when the computing device generates the second counting data based on the second image data, the display portion displays the second image data and/or the second counting data.
